(19) United States
(12) Patent Application Publication
(10) Pub. No.: US 2020/0411188 A1
(43) Pub. Date: Dec. 31, 2020

(54) IMPROVEMENTS IN OR RELATING TO PSYCHOLOGICAL PROFILES

(71) Applicant: IESO DIGITAL HEALTH LIMITED, Cambridge (GB)

(72) Inventors: Ana Maria Ferreira Paradela Catarino WINGFIELD, Cambridge (GB); Alan James MARTIN, Cambridge (GB); Andrew BLACKWELL, Cambridge (GB); Jonathan Matthew FAWCETT, Cambridge (GB)

(21) Appl. No.: 16/978,257

(22) PCT Filed: Mar. 6, 2019
(86) PCT No.: PCT/GB2019/050616

§ 371 Date: Sep. 4, 2020

(30) Foreign Application Priority Data
Mar. 6, 2018 (GB) 1803604.6
May 17, 2018 (GB) 1808020.0

(51) Int. Cl. G16H 50/20; G16H 15/00; G16H 10/20; A61B 5/16; G16H 20/70; G16H 50/30

(52) U.S. Cl. CPC G16H 50/20; G16H 15/00; G16H 50/30; A61B 5/165; G16H 20/70; G16H 10/20

(57) ABSTRACT

A computer-implemented method of assigning a treatment protocol to a patient, comprising the steps of: obtaining a plurality of patient profile data points relating to the patient at an initial stage of a psychotherapy process; comparing each patient profile data point with the corresponding data point for each one of a plurality of reference profiles; selecting from the plurality of reference profiles the reference profile to which the patient profile data most closely fits, in order to obtain a prediction of the psychological condition of the patient; assigning a treatment protocol to the patient based on the prediction of the psychological condition of the patient; wherein the plurality of reference profiles are determined by modelling a reference dataset comprising patient profile data relating to each of a plurality of other patients.

### FIELD OF THE INVENTION

The present application relates among other things to methods for use by a computer-based system for profiling the symptoms of a psychological condition, determining subtypes of a psychological condition, and/or assigning a patient to a psychotherapy treatment protocol.

### BACKGROUND OF THE INVENTION

Common mental health disorders including depression and anxiety are characterized by intense emotional distress, which affects social and occupational functioning. About one in four adults worldwide suffer from a mental health problem in any given year. In the US, mental disorders are associated with estimated direct health system costs of $201 billion per year, growing at a rate of 6% per year, faster than the gross domestic product growth rate of 4% per year. Combined with annual loss of earnings of $193 billion, the estimated total mental health cost is at almost $400 billion per year. In the UK mental health disorders are associated with service costs of £22.5 billion per year and annual loss of earnings of £26.1 billion.

Mental health disorders, including depression, do not present homogeneously, in that any particular patient may experience one or more of a number of possible symptoms, and to a greater or lesser extent than for other patients. Therefore at presentation a patient may display a complex profile of symptoms varying in number, duration, and severity.

Furthermore, often a variety of treatment protocols may be available for a particular mental health disorder. In relation to depression, the different treatment approaches may include the provision of information to the patient, the prescription of psychotropic medication, or the provision of psychotherapy (e.g. cognitive behavioral therapy (CBT)), via either face-to-face sessions of therapy delivered in person between a therapist and a patient, or via online therapy, including internet-enabled cognitive behavioral therapy (IECBT). Each of these treatment approaches itself may include a number of possible variants, and each may be provided in isolation or in combination with each other to give a treatment protocol. Different treatment protocols would be expected to be effective to a greater or lesser extent in improving the symptoms of a patient (or group of patients) depending on their particular presenting symptoms, their aetiology and their severity. Therefore attempts have been made previously to categorise or classify mental health disorders into particular subtypes or severities, in order that more appropriate treatment protocols may be provided to patients falling within a particular group.

However, these previous attempts to categorise mental health disorders into subtypes are of limited utility, for example because different disorders may overlap in terms of the symptoms with which they are typically associated. The main classification systems in use rely on some fairly arbitrary decisions about symptoms, and some of the subtypes can appear difficult to distinguish and may not form distinct categories. Furthermore, no reliable classificatory system has yet emerged for depression that has proven strongly predictive of response to treatment.

Therefore due to the currently arbitrary and subjective aspects of mental health disorder subtyping, particularly depression subtyping, the allocation of a patient to a particular treatment protocol may also be considered to be arbitrary or subjective.

Furthermore, no reliable method has been described to date to demonstrate which symptoms of depression are the most clinically significant, and how the different symptoms interact. The studies performed to date do not provide conclusive evidence for the existence of depressive symptom dimensions or symptomatic subtypes (van Loo et al., ‘Data-driven subtypes of major depressive disorder: a systematic review’, BMC Medicine 2012, 10:156).

The methodologies of the current classification systems use a patient questionnaire with an arbitrary numerical threshold, which weights the different symptoms of depression equally, although, in fact, patterns of symptoms may be more subtle and more important. By way of further example, when making a diagnosis of a Major Depressive Episode (MDE) within the DSM-IV framework, only “depressed mood” (mood) or “loss of interest or pleasure in nearly all activities” (anhedonia) are considered to be essential symptoms required for diagnosis, thereby effectively ignoring, or diminishing the significance of, other potentially important symptoms such as fatigue, sleep disturbance, anxiety, and neurocognitive dysfunction. A method to objectively distinguish which symptoms are the most important, and how different symptoms interact, could be used to objectively determine which symptoms are of core clinical significance with respect to both assessment and treatment.

For these reasons, a new approach is required to improve, augment or assist with initial assessment of a patient with a mental health disorder, the categorisation of a mental health disorder into subtypes, the allocation of a patient with a mental health disorder to a particular treatment protocol, and the prioritisation of assessment and treatment to particular symptoms of a mental health disorder.

### SUMMARY OF THE INVENTION

According to a first aspect of the present invention, there is provided a computer-implemented method of assigning a treatment protocol to a patient, comprising the steps of:

obtaining a plurality of patient profile data points relating to the patient at an initial stage of a psychotherapy process; comparing each patient profile data point with the corresponding data point for each one of a plurality of reference profiles;

selecting from the plurality of reference profiles the reference profile to which the patient profile data most closely fits, in order to obtain a prediction of the psychological condition of the patient;

assigning a treatment protocol to the patient based on the prediction of the psychological condition of the patient;

wherein the plurality of reference profiles are determined by modelling a reference dataset comprising patient profile data relating to each of a plurality of other patients. Thus the method may improve the assignment of a treatment protocol to a patient, by predicting the psychological condition of which the patient suffers. By making a prediction of the psychological condition of the patient, the treatment protocol selected from a number of possible treatment protocols may be the most appropriate protocol for the patient's condition. In other words, the method provides a form of personalised medicine. Thus the method may lead to increased likelihood of improvement or recovery of the patient, i.e. a better outcome for the patient. The method may also lead to decreased costs to the therapy provider or service, as the psychotherapy process is likely to be more efficient.

Furthermore, the method may comprise the additional step of treating the patient according to the assigned treatment protocol.

The plurality of patient profile data points may be considered inputs to the method; each patient profile data point may comprise non-binary data. For example, each patient profile data point may comprise one option selected from a plurality of possible options, e.g. the patient data points may comprise a numerical value selected from a possible range, e.g. a score of 0, 1, 2 or 3 etc. Alternatively the patient profile data points may comprise a combination of non-binary and binary data.

The plurality of patient profile data points may comprise data relating to one or more symptoms of the patient. For example, the plurality of patient profile data points may comprise symptoms self-reported by the patient, symptoms measured by a therapist, or symptoms determined by one or more devices such as a computer interface or mobile electronic device. The plurality of patient profile data points may comprise remote data, in other words data not measured directly from the body of the patient.

The plurality of patient profile data points may comprise multiple data points indicative of the strength of a patient's agreement with multiple statements. For example the plurality of patient profile data points may comprise item scores derived from a standardised psychology questionnaire. Examples of such questionnaires are the PHQ-9 or GAD-7 questionnaires. Standardised psychology questionnaires provide a convenient, straightforward and standardised way in which patients may report their symptoms. By this method patients may easily report the symptoms of their psychological condition. Standardised psychology questionnaires provide a number of items/questions, each relating to a particular symptom, for each of which a patient is typically requested to give a score from a provided range in order to illustrate either the frequency or severity with which they are experiencing a given symptom. Thus standardised psychology questionnaires provide a rich source of qualitative data relating to patient(s) symptoms; the qualitative nature of the data may be difficult for therapists to process objectively, meaning that standard therapy methods ignore a large amount of the available data and may not therefore make accurate predictions about the patient's condition.

The plurality of reference profiles may comprise states determined by modelling the reference dataset using a Hidden Markov Model (HMM). An HMM may be used to reveal a plurality of hidden states within the reference dataset, each hidden state may comprise a profile, i.e. a multimodal, multifactorial or multidimensional solution space. The patient may be suffering from a mental health disorder, wherein the disorder optionally may comprise a disorder selected from the group consisting of (1) depression, (2) mixed anxiety and depression, and (3) generalized anxiety disorder.

Further, the disorder may comprise a disorder selected from the group consisting of agoraphobia, health anxiety, obsessive compulsive disorder (OCD), post-traumatic stress disorder (PTSD), panic disorder, social anxiety disorder and specific phobia.

The disorders ‘depression’, ‘mixed anxiety and depression’ and ‘generalized anxiety disorder’, ‘agoraphobia’, ‘health anxiety’, ‘obsessive compulsive disorder (OCD)’, ‘post-traumatic stress disorder (PTSD)’, ‘panic disorder’, ‘social anxiety disorder’ and ‘specific phobia’ are examples of condition labels traditionally assigned to patients by therapists and other healthcare providers. For example, a patient may present to a therapy service having been told by their general practitioner that they are suffering from depression. Therefore a patient who is suffering from a mental health disorder, or a particular named mental health disorder, means a patient who has been labelled as such following a traditional (subjective) diagnosis.

For example, depression can be characterized by a wide range of psychological and physical symptoms, and the heterogeneity of depression in the current (largely subjective) classification system remains a point of discussion amongst clinicians. Theoretically driven subtypes of depression such as melancholic, atypical and psychotic depression seem to have limited clinical applicability, while data-driven approaches for symptom dimension analysis and subtyping remain scarce.

In contrast with this, the invention described herein reveals ‘hidden’ states and characterizes a patient's condition by taking an objective approach, looking at intensity of symptoms relative to each other, thereby providing an improvement to the more subjective condition labels assigned to patients during traditional diagnosis. This is advantageous because inter-rater reliability in terms of diagnosis is known to be low across therapists, and other healthcare providers. Hence relying on traditional diagnosis alone potentially results in a high incidence of misdiagnosis of patients, and therefore a high incidence of inappropriate, or suboptimal, treatment being provided to patients.

The prediction of the psychological condition of the patient may comprise a subtype of depression, and/or a severity of depression. Herein, subtype may be used to mean a subtype of depression with a particular combination of presenting symptoms, a subtype of depression with a particular aetiology, a subtype of depression displaying a particular response to treatment, and/or a subtype of depression with a particular severity. The subtypes of depression of the invention may correspond or overlap with previously-described (known) subtypes of depression, or they may be new subtypes not previously defined.

Furthermore, the prediction of the psychological condition of the patient may comprise a prediction of a type or subtype of any mental health disorder/condition, and/or a severity of any mental health disorder/condition. Herein, type or subtype may be used to mean a type or subtype of a mental health disorder with a particular combination of presenting symptoms, a type or subtype of a mental health disorder with a particular aetiology, a type or subtype of a mental health disorder displaying a particular response to treatment, and/or a type or subtype of a mental health disorder with a particular severity. The types or subtypes of mental health disorders revealed by the invention may correspond or overlap with previously-described (known) types or subtypes of mental health disorders, or they may be new types or subtypes not previously defined.

The psychotherapy process in accordance with any aspect of the invention may comprise internet-enabled cognitive behavioural therapy.

In accordance with a second aspect of the present invention, there is provided a computer-implemented method of determining subtypes of a psychological condition comprising:

obtaining patient profile data relating to each of a plurality of patients;

using a Hidden Markov Model to find a plurality of reference profiles in the patient profile data;

wherein each reference profile describes a subtype of the psychological condition.

The computer-implemented method of determining subtypes of a psychological condition may further comprise: obtaining a plurality of patient profile data points relating to the patient at an initial stage of a psychotherapy process; comparing each patient profile data point with the corresponding data point for each one of the plurality of reference profiles; selecting from the plurality of reference profiles the reference profile to which the patient profile data most closely fits, in order to obtain a prediction of the psychological condition of the patient; and assigning a treatment protocol to the patient based on the prediction of the psychological condition of the patient.

The computer-implemented method of determining subtypes of a psychological condition may alternatively further comprise: obtaining a plurality of patient profile data points relating to a patient at an initial stage of a psychotherapy process; comparing each patient profile data point with the corresponding data point for each one of the plurality of reference profiles; selecting from the plurality of reference profiles the reference profile to which the patient profile data most closely fits in order to obtain an output predicting a characteristic of a condition of the patient; and causing the system to take one or more actions relating to the psychotherapy process, wherein the one or more actions are selected based on the output.

The computer-implemented method of determining subtypes of a psychological condition may alternatively further comprise: assigning each of the plurality of reference profiles to a family of reference profiles based on the probability of transition between each of the plurality of reference profiles; identifying core symptoms of the family using a network analysis of individual dimensions of the patient profile data for said family; and designing a treatment protocol to target the core symptoms for improved analysis and treatment of psychological conditions.

In accordance with a third aspect of the present invention there is provided a computer-implemented method of determining families of subtypes of a psychological condition comprising:

obtaining first patient profile data relating to each of a plurality of patients at a first stage of a treatment process;

obtaining second patient profile data relating to each of the plurality of patients at a second stage of a treatment process;

obtaining combined patient profile data by combining the first patient profile data and the second patient profile data;

using a Hidden Markov Model to find a plurality of reference profiles in the combined patient profile data;

using the Hidden Markov Model to further find a probability of transition between each of the plurality of reference profiles;

assigning each of the plurality of reference profiles to a family of reference profiles based on the probability of transition between each of the plurality of reference profiles;

wherein each reference profile describes a subtype of the psychological condition.

Optionally, further patient profile data relating to each of a plurality of patients at one or more further stages of a treatment process may also be obtained. For example, patient profile data relating to each of a plurality of patients at all stages of a treatment process may be obtained. The combined patient profile data may be obtained by combining the first patient profile data, the second patient profile data, and the further patient profile data. Thereby, the combined patient profile data may comprise patient profile data relating to all stages of a treatment process.

The patient profile data, the first patient profile data, the second patient profile data, and/or the further patient profile data may comprise data relating to one or more symptoms of each of the plurality of patients. For example, the patient profile data, the first patient profile data, the second patient profile data, and/or the further patient profile data may comprise data derived from a standardised psychology questionnaire, optionally wherein the standardised psychology questionnaire is selected from the HQ-9 questionnaire or the GAD-7 questionnaire. Standardised psychology questionnaires provide a rich source of qualitative data relating to patients symptoms, thus subtypes of a psychological condition may be objectively determined by the method, using the maximal amount of available data.

The psychological condition in accordance with any aspect of the invention may comprise depression.

In accordance with a further aspect of the invention there is provided a method for use by a computer-based system for providing psychotherapy, the method comprising:

obtaining a plurality of patient profile data points relating to a patient at an initial stage of a psychotherapy process;

comparing each patient profile data point with the corresponding data point for each one of a plurality of reference profiles;

selecting from the plurality of reference profiles the reference profile to which the patient profile data most closely fits in order to obtain an output predicting a characteristic of a condition of the patient; and

causing the system to take one or more actions relating to the psychotherapy process, wherein the one or more actions are selected based on the output;

wherein the plurality of reference profiles are determined by modelling a reference dataset comprising patient profile data relating to each of a plurality of other patients.

One or more actions taken by the system may include (1) assigning a treatment protocol to the patient, (2) providing the output as an input to a system performing ‘digital triage’.

In some embodiments of any aspect of the invention each step of the method may be performed in a step-wise manner. It will be understood by the person skilled in the art that in other embodiments of any aspect of the invention a number of steps of the method may be performed in any practical order. Alternatively, two or more steps may be conducted contemporaneously.

In accordance with a further aspect of the invention there is provided a data processing apparatus/device/system comprising means for carrying out the steps of the method according to any of the preceding claims.

In accordance with a further aspect of the invention there is provided a computer program comprising instructions which, when the program is executed by a computer, cause the computer to carry out the steps of the method according to any of the preceding claims.

In accordance with a further aspect of the invention there is provided a computer-readable storage medium comprising instructions which, when executed by a computer, cause the computer to carry out the steps of the method according to any of the preceding claims.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a flow diagram of a computer-implemented method of the present disclosure.

FIG. 2 illustrates an example of a number of depression states (reference profiles) as modelled by the methods of the invention, as determined by the profiles of patient responses to the PHQ-9 questionnaire. FIG. 2A shows profiles (states) 1-6; FIG. 2B shows profiles (states) 7-10. The X-axis represents each item (question, symptom) from the PHQ-9 questionnaire, and the Y-axis represents the mean numerical score attributed to that question for all patients allocated to that disease state by the algorithm of the invention.

FIG. 3 illustrates all 10 reference profiles (depression states) as shown in FIG. 2, presented on the same figure for ease of comparison

FIG. 4 illustrates the probabilities of transitions over time between the depression states (reference profiles) as depicted and numbered in FIGS. 2 and 3, for patients from the start of their treatment to the end. Transition probabilities of less than 15% are not shown. The thickness of the arrows indicates the probability of a transition between two particular states (profiles) occurring. Increasing severity of disease states is represented on the Y-axis, a typical threshold of clinical significance (PHQ-9=10) is shown by a dashed line.

FIG. 5 illustrates a network analysis of symptoms derived from responses to the PHQ-9 questionnaire showing the centrality of symptoms, derived from data gathered from 5177 patients. Each of the nine questions/items in the questionnaire is represented by a node (designated FPHQ.Q1 to FPHQ.Q9), and the edges (lines) between them show the inter-relatedness of the questions, and therefore the symptoms they represent. The thickness of the connecting edges correlates to the degree of relatedness between the nodes. It can be seen that in this example, the strongest nodes are question 2 (FPHQ.Q2) and question 4 (FPHQ.Q4).

FIG. 6 illustrates a similar network analysis of symptoms derived from combined responses to both the PHQ-9 (nodes designated FPHQ.Q1 to FPHQ.Q9) and GAD-7 (nodes designated FGAD.Q1 to FGAD.Q7) questionnaires showing the centrality of symptoms, derived from data gathered from 5177 patients. It can be seen that in this example, the strongest nodes are PHQ-9 question 2 (FPHQ.Q2) and GAD-7 question 5 (FGAD.Q5), whilst the node with highest degree of closeness is PHQ-9 question 7 (FPHQ.Q7).

FIG. 7 illustrates PHQ-9 item change over time, demonstrating that certain symptoms (PHQ-9 item scores) show less change than others over a course of treatment.

FIG. 8 illustrates the transitions over time between reference profiles (representing depression states), as depicted in FIG. 9, for patients over an entire course of treatment (up to 10 sessions, session number on X-axis). Each graph corresponds to a different starting state (state as determined at the first treatment session), and each differentially shaded area represents the proportion of patients in a given state/allocated to a particular reference profile (proportion of patients belonging to the starting state on Y-axis). Changes to the proportion of patients in a given state can be visualized over the course of treatment. For example for starting state 5, the proportion of patients in this state decreases with time (area of darkest grey shading decreases as treatment session number increases), as patients transition to other (mostly less severe) states (area of lighter grey shading increases as treatment session number increases).

FIG. 9 illustrates an example of a number (n=7) of reference profiles (representing depression states) as modelled by the methods of the invention, as determined by the profiles of patient responses to the PHQ-9 questionnaire. The X-axis represents each item (question, symptom) from the PHQ-9 questionnaire, and the Y-axis represents the mean numerical score attributed to that question for all patients allocated to that disease state by the algorithm of the invention.

### DETAILED DESCRIPTION OF THE INVENTION

The present disclosure relates to computer-implemented methods for profiling the mental health disorder of a patient, and thereby allocating that patient to an appropriate treatment protocol.

A variety of different treatment options or therapies are available for the treatment of mental health disorders (mental health conditions; psychological conditions). The selection of the most appropriate treatment protocol for a particular patient, in other words the protocol most likely to result in improvement or recovery for that patient, relies on both a reliable diagnosis of the patient's condition, and also an understanding of the treatment protocol most likely to result in improvement for that condition. Both of these factors in turn rely on the ability to differentiate between closely related conditions, or between subtypes of a particular condition.

### Depression

Depression (clinically significant depression; major depression; major depressive disorder (MDD)) is an example of a mental health disorder, characterised by persistent low mood and/or loss of pleasure in most activities (anhedonia) and a range of associated emotional, cognitive, physical, and behavioural symptoms, including but not limited to: fatigue/loss of energy, feelings of worthlessness or excessive or inappropriate guilt, recurrent thoughts of death, suicidal thoughts, or actual suicide attempts, diminished ability to think/concentrate, or indecisiveness, psychomotor agitation or retardation, insomnia or alternatively hypersomnia, significant appetite loss and/or weight loss. Mild (sub-threshold) depressive symptoms (e.g. dysthymia; persistent subthreshold depressive symptoms) are also recognised as distressing and disabling if present for extended periods of time (months/years).

### Diagnosis of Depression

Two commonly used major classification systems for depression are available, derived from DSM-IV-TR (‘Diagnostic and Statistical Manual of Mental Disorders’, published by the American Psychiatric Association; since superseded by DSM-5) and ICD-10 (‘International Statistical Classification of Diseases and Related Health Problems 10th Revision’, published by the World Health Organisation). The latter system is typically used in European countries, while the former is currently used in the US and many other non-European nations. Both systems may be used by clinicians in the UK. The two classification symptoms define depression in convergent, but non-identical, ways.

For example, ICD-10 defines three main depressive symptoms (depressed mood, anhedonia, and reduced energy), of which two should be present to determine depressive disorder diagnosis. Furthermore, ICD-10 also requires a total of at least four out of ten depressive symptoms to be present for a formal diagnosis of depression to be made.

In contrast, only one of two main symptoms (depressed mood, anhedonia) are considered to be essential requirements for the diagnosis of a Major Depressive Episode (MDE) in DSM-IV, along with the presence of a total of five symptoms out of a possible nine (in addition to depressed mood and/or anhedonia, the other symptoms taken into account by DSM-IV are disturbed sleep, appetite and/or weight changes, fatigue or loss of energy, agitation or slowing of movements, poor concentration or indecisiveness, feelings of worthlessness or excessive or inappropriate guilt, suicidal thoughts or acts).

Both systems require the symptoms to have been present for at least the two preceding weeks, and of sufficient severity to cause clinically significant distress or impairment in social, occupational, or other important areas of functioning. The presence and severity of symptoms may be assessed using a depression questionnaire.

### Mental Health Disorder Subtyping

Taking depression as an example of a mental health disorder, various attempts have been made to classify depressive disorders into subtypes and/or severity levels.

Due to the plurality of possible symptoms of depression, the sometimes mutually-exclusive nature of those symptoms, and the fact that each patient may experience those symptoms to a greater or lesser extent (in comparison with other patients, or over time), depression can be seen to be a heterogeneous disorder. The ability to effectively treat depression with an appropriate treatment protocol may require the need to better define the severity and/or subtype of depression experienced by a particular patient.

### Severity

The two classification systems ICD-10 and DSM-IV classify clinically important depressive episodes as mild, moderate and severe based on the number, type and severity of symptoms present and degree of functional impairment experienced by the patient.

The current UK NICE Guidelines for diagnosing depression, based on the DSM-IV or DSM-5 criteria, divide severity into three categories: severe, moderate and mild. In addition, subthreshold depression has its own definition. ‘Severe’ depression means several symptoms are present in excess of those required to make the diagnosis. Some symptoms would be expected to be severe and markedly interfere with functioning. ‘Moderate’ depression means symptoms or functional impairment lie between the levels for severe and mild. Some symptoms would be expected to be marked. ‘Mild’ depression means few, if any, symptoms in excess of the five required to make a diagnosis are present, and the patient is experiencing only minor functional impairment.

Due to the fact that the minimum number of symptoms required for a diagnosis of clinically-significant depression is higher when using the DSM-IV or DSM-5 classification systems (five symptoms), than the ICD-10 system (four symptoms), the threshold for mild depression is higher when using either DSM system.

### Sub-Threshold Depression

Both DSM-IV and ICD-10 include the category of ‘dysthymia’, which consists of depressive symptoms that are subthreshold for (major) depression but that persist (by definition in ICD-10 for more than 2 years). There appears to be no empirical evidence that dysthymia is distinct from subthreshold depressive symptoms in general, apart from duration.

In DSM-5, what was referred to as dysthymia in DSM-IV now falls under the category of ‘persistent depressive disorder’, which includes both chronic major depressive disorder and the previous dysthymic disorder. An inability to find scientifically meaningful differences between these two conditions led to their combination into a single category.

Thus it can be seen that the diagnosis of depression, or particular severities of depression, relies on the application of (somewhat) arbitrary and divergent thresholds. Whether a patient is deemed to need treatment, and the form that treatment takes, may be determined, at least in part, by the severity of depression diagnosed. Therefore it is advantageous that the diagnosis thresholds are objectively determined.

With respect to diagnosis in individuals with milder symptoms, the application of an arbitrary threshold may result in that individual being excluded from active treatment; depressive symptoms below the DSM and ICD-10 threshold criteria can be distressing and disabling if persistent, and these patients may benefit from the provision of appropriate treatment protocols.

### Subtypes

In addition to classifying depressive disorders into severity levels, various attempts have also been made to classify depression into subtypes. This has been in response to the heterogeneous nature of the condition in terms of e.g. presenting symptoms and/or aetiology. Despite attempts to link the symptoms of depression with its aetiology, including neurobiological, genetic and psychological studies, no reliable classification system has emerged that links symptoms presentation to either the underlying aetiology or has proven strongly predictive of response to treatment.

Historically, a number of subtypes have been proposed, including reactive and endogenous depression, melancholia, atypical depression, depression with a seasonal pattern/seasonal affective disorder and dysthymia, as well as duration and course of the disorder (for example, single episode, recurrent, presence of residual symptoms).

Within DSM-IV-TR, severe major depression (MDD; clinically-significant depression; depression) may be categorised further into subtypes as: without or with psychosis (psychotic depression), and may further include melancholia, atypical features, catatonia, depression with a seasonal pattern (seasonal affective disorder) or post-partum onset. However, these subtypes do not form distinct categories, and do not necessarily predict response to treatment, either per se or of a particular type.

Some studies have attempted to define depression into subtypes empirically, as opposed to by subjective observation. For example Drysdale et al., ('Resting-state connectivity biomarkers define neurophysiological subtypes of depression', Nature Medicine volume 23, pages 28-38 (2017)) attempted to define depression into four subtypes by imaging patterns of dysfunctional neuronal connectivity in the brain using functional magnetic resonance imaging (fMRI). This approach is inconvenient to the patient, and time-consuming and extremely expensive to perform, and additionally, it is unclear how the different subtypes designated map to the symptoms experienced by patients and/or if each subtype may be predictive of response to treatment.

The above outlined conventions and methods for diagnosing and/or sub-typing depression demonstrate that the current classification systems are variable, likely to result in differential diagnoses between systems, and have no proven link to treatment outcome.

### Questionnaires

A patient's initial assessment is typically conducted by a clinician, who may take into account the patient's medical history, personal circumstances, and particularly current symptoms, when attempting to diagnose the presence of a psychological condition or mental health disorder. In order to assess a patient's symptoms, a clinician may utilise one or more standardised psychological questionnaire(s), such as the PHQ-9 questionnaire for depressive disorders, or the GAD-7 questionnaire for anxiety disorders. Each questionnaire poses a number of questions/items relating to particular symptoms (e.g. nine for PHQ-9; seven for GAD-7), to which the patient responds with a score of between 0 and 3 depending on their self-assessment of the severity/frequency of their symptoms. Therefore the clinician is provided by the questionnaire responses with a multimodal, multidimensional solution space, from which to make an assessment of the patient's particular psychological condition and its severity. Despite the rich and complex information potentially provided by the questionnaire responses, a typical way in which a clinician would make their assessment of the patient would be to sum the individual scores given in response to each question. If the sum total is greater than a pre-determined threshold the patient would be nominally deemed to meet ‘caseness’, i.e. to exhibit clinically significant symptoms. Further thresholds may be used to define severities. This approach of applying numerical threshold(s) to the questionnaire data is disadvantageous because it largely disregards the complexity of the data provided, assumes that the different symptoms are equivalent, and applies an arbitrary cut-off to a varying spectrum of symptoms on a severity continuum, thereby potentially making an incorrect or incomplete assessment of the patient. For example, two patients may present with divergent symptoms, but if the sum total was the same for each patient, current methodologies may treat them identically.

### Patient Health Questionnaire (PHQ-9)

PHQ-9 is a nine item self-administered questionnaire that detects the presence and/or severity of depression (see Kroenke, K., et al. The PHQ-9: validity of a brief depression severity measure. J Gen Intern Med, 16, p. 606, 2001). It has been specifically designed for use in primary care. The questions/items/symptoms assessed by the PHQ-9 questionnaire are set out in Table 1. For each item a patient is required to give a score between 0 and 3, indicating the frequency with which they experienced that item/symptom in the preceding two weeks, where 0=Not at all, 1=Several days, 2=More than half the days, 3=Nearly every day.

TABLE 1
PHQ-9 items/symptoms

| Item No. | Symptom |
|---|---|
| 1 | Little interest or pleasure in doing things |
| 2 | Feeling down, depressed, or hopeless |
| 3 | Trouble falling or staying asleep, or sleeping too much |
| 4 | Feeling tired or having little energy |
| 5 | Poor appetite or overeating |
| 6 | Feeling bad about yourself - or that you are a failure or |
| | have let yourself or your family down |
| 7 | Trouble concentrating on things, such as reading the |
| | newspaper or watching television |
| 8 | Moving or speaking so slowly that other people could have |
| | noticed? Or the opposite - being so fidgety or restless |
| | that you have been moving around a lot more than usual |
| 9 | Thoughts that you would be better off dead or of hurting |
| | yourself in some way |

PHQ-9 score totals typically used to correspond to depression severity are set out in Table 2.

TABLE 2
PHQ-9 scores and depression severity

| PHQ-9 score total | Depression severity |
|---|---|
| 0-4 | No depression |
| 5-9 | Mild depression |
| 10-14 | Moderate depression |
| 15-19 | Moderately severe depression |
| 20-27 | Severe depression |

Other suitable alternatives to the PHQ-9 questionnaire for use in diagnosing depression are known, including Hospital Anxiety and Depression Scale (HADS), and Beck Depression Inventory-II (BDI-II).

### Generalised Anxiety Disorder (GAD 7) Questionnaire

The Generalised Anxiety Disorder (GAD 7) is a seven item self-administered questionnaire that is designed as a screening and severity measure for generalised anxiety disorder (GAD). The GAD-7 also has moderately good operating characteristics for three other common anxiety disorders, namely panic disorder, social anxiety disorder and post-traumatic stress disorder (see Spitzer, R. L., et al. (2006). A Brief Measure for Assessing Generalized Anxiety Disorder: The GAD-7. Arch Intern Med. 166, 1092-1097). The questions/items/symptoms assessed by the GAD-7 questionnaire are set out in Table 3, and the total GAD-7 scores typically used to correspond to anxiety severity are set out in Table 4. The criteria for attributing a particular score to each GAD-7 item are identical to those for PHQ-9.

TABLE 3
GAD-7 items/symptoms

| Item No. | Symptom |
|---|---|
| 1 | Feeling nervous, anxious or on edge |
| 2 | Not being able to stop or control worrying |
| 3 | Worrying too much about different things |
| 4 | Trouble relaxing |
| 5 | Being so restless that it is hard to sit still |
| 6 | Becoming easily annoyed or irritable |
| 7 | Feeling afraid as if something awful might happen |

TABLE 4
GAD-7 scores and anxiety severity

| GAD-7 score total | Anxiety severity |
|---|---|
| 0-4 | No anxiety |
| 5-9 | Mild anxiety |
| 10-14 | Moderate anxiety |
| 15-21 | Severe anxiety |

Other suitable alternatives to the GAD-7 questionnaire for use in diagnosing anxiety are known, including Hospital Anxiety and Depression Scale (HADS), Hamilton Anxiety Scale (HAM-A), Obsessive Compulsive Inventory (OCI), Impact of events scale—revised (IES-R), Agoraphobia Mobility Inventory (AMI), Social Phobia Inventory (SPI), Panic disorder severity scale (PDSS) and health anxiety inventory (HAI).

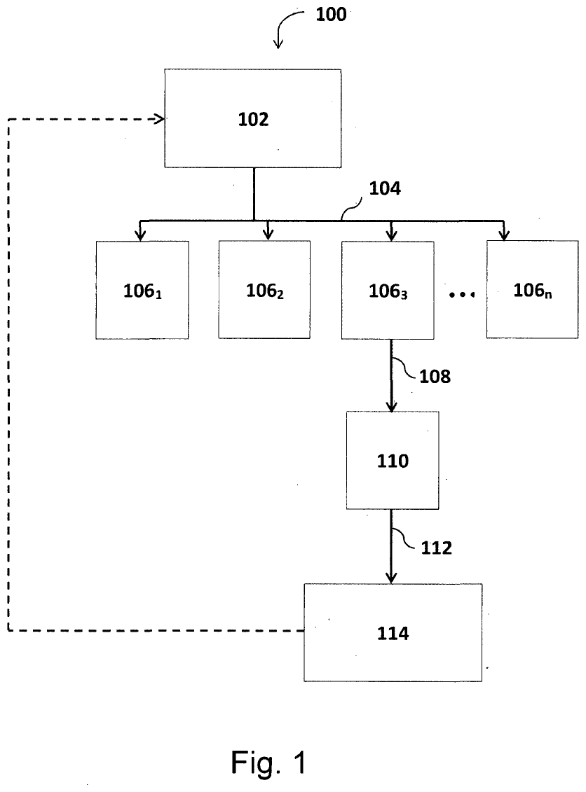

FIG. 1 illustrates a flow diagram of a computer-implemented method 100 of the present disclosure. First, patient profile data (a plurality of patient profile data points) 102 is obtained as inputs.

Exemplary patient profile data points include patient responses to one or more standardised psychological questionnaire(s) Exemplary standardised psychological questionnaire(s) include, but are not limited to, PHQ-9, GAD-7, HADS, HAM-A, BDI-II, OCI, IES-R, AMI, SPI, PDSS and HAI.

The patient profile data may be directly inputted by the patient into a computer or computer program, or the patient profile data may be provided verbally or in writing to another person, for example a clinician, therapist, receptionist or other therapy service personnel member, who then inputs the data into a computer or computer program. Alternatively, the patient profile data may comprise information remotely or passively collected about a patient's symptoms, for example by a mobile computing device.

Referring again to FIG. 1, after the patient profile data inputs 102 are collected, a comparison 104 is made between each patient profile data point and a corresponding data point from each of a plurality of reference profiles 106ₙ. The corresponding data points may comprise calculated values such as means. The plurality of reference profiles 106 may comprise states outputted by a Hidden Markov Model used to model a reference dataset comprising patient profile data from a plurality of other patients.

After the patient profile data 102 are compared 104 with the reference profiles 106ₙ, the reference profile 106 which provides the best fit to the patient profile data is selected 108. The selection 108 of a reference profile provides a prediction of the psychological condition of the patient (or output) 110.

Once the output or prediction of the psychological condition of the patient 110 has been obtained, the patient is assigned 112 to a particular treatment protocol (an action) 114. Thus a treatment protocol appropriate for the condition of the patient may be provided.

Once the output or prediction of the psychological condition of the patient 110 has been obtained, a particular action is taken by the method or system, for example the patient is assigned 112 to a particular treatment protocol (an action) 114. Thus a treatment protocol appropriate for the condition of the patient may be provided.

Therefore, treatment protocols described herein may be considered non-limiting examples of treatment protocols, and further may be considered non-limiting examples of actions 114 that may be taken by the methods described.

Following provision of the treatment protocol, the method may be repeated, i.e. patient profile data inputs 102 may be collected at a second or subsequent time point, in order that the psychological condition of the patient may be predicted again, in order to determine the effectiveness of the treatment protocol.

### Treatment Protocol Options

Various treatment protocol 114 options for depressive illnesses are available to the clinician; these may include one or more of: watchful waiting, guided self-help, traditional cognitive behavioral therapy (CBT), computerised CBT, internet-enabled CBT (IECBT), exercise, psychological interventions (brief, standard or complex), medication, social support, combined treatments, and/or electroconvulsive therapy (ECT).

Online therapy, including internet-enabled cognitive behavioral therapy (IECBT), offers significant advantages over standard care. Internet-enabled cognitive behavioral therapy (IECBT) is a type of high-intensity online therapy used within an Improving Access to Psychological Therapies (IAPT) program. Within IAPT using IECBT, patients are offered weekly one-to-one sessions with an accredited therapist, similar to face-to-face programs, whilst also retaining the advantages of text-based online therapy provision including convenience, accessibility, increased disclosure and shorter waiting times. The improvement rate for patients treated with IECBT is significantly higher than for severity-matched patients treated with standard care.

Variations in the treatment protocol 114 within IECBT may include the frequency of one-to-one or face-to-face meetings, the frequency of asynchronous messaging in between sessions, the potential need for psychotropic medication(s), or treatment by a particular therapist as part of the treatment protocol.

The assignment of the most appropriate treatment protocol to a particular patient is assisted by meaningful classification or subtyping of the patient's condition.

For example, a patient for whom the prediction of psychological condition is of a mild severity subtype, i.e. with a high probability of correlating with a state falling below the traditional diagnosis threshold, may be assigned to a treatment protocol 114 with fewer one-to-one or face-to-face meetings, than for a patient for whom the prediction of psychological condition is more severe.

In addition, a patient for whom the prediction of psychological condition is of a particular subtype, for example as defined by a high probability of correlating with a particular family of related reference profiles (states), may be offered a treatment protocol appropriate to that family, wherein the treatment protocol is known or predicted to be effective for that subtype or family. A particular treatment protocol may be designed to target the symptoms or groups of symptoms of greatest importance in a particular family of profiles.

For example, the methods disclosed herein were used to identify three distinct subtypes of depression: Somatic depression, Cognitive depression, and Hybrid depression. Each subtype of depression correlated with a particular family of related reference profiles (states). Thereby, each subtype of depression was correlated with particular symptoms. For example, somatic depression is characterized by high intensity of physical symptoms, including tiredness, difficulties sleeping and changes in appetite. Cognitive depression is characterized by high intensity of symptoms such as low mood, low self-esteem and high suicidal ideation. More severe hybrid depression is characterized by high intensity of both physical and psychological symptoms.

The symptom profiles, relatedness of and underlying nature of the subtypes elucidated using the symptom profiler may be useful to tailor treatment to particular symptom profile(s) or subtype(s). The symptom profiler may thus be used to assist in the provision of personalized medicine.

The symptoms of greatest importance in a particular subtype, or family of subtypes, of a psychological condition (the core symptoms) may furthermore be determined by performing network analysis on the individual dimensions of the patient profile data, for example the items of a standard psychological questionnaire. The symptom(s) (item(s), question(s), node(s)) of greatest centrality may be selected, and a treatment protocol may be designed or provided in order to target those particular symptom(s). In that way, the core symptom(s) would be directly treated, and due to their correlation with the core symptom(s) the related/connected symptoms would be expected to be indirectly treated. Thereby the methods of the present disclosure provide improved analysis and treatment of psychological conditions.

The one or more actions may comprise allocating the patient to one of a plurality of therapists. The allocation may be based at least in part on a prediction of the psychological condition of the patient (or output) 110 and on data describing the performance of the therapist in relation to the psychological condition. Thus, the method may match patients with therapists who are likely to provide more effective and/or efficient psychotherapy to the patient. For example, patients that are predicted to belong to a given reference profile at initial assessment may be allocated to therapists who have been determined to provide more effective treatment to patients of that reference profile. Thus, the method may use therapist resources in an optimal way to provide the best and most cost effective treatment. The allocation may also be based on further data (e.g. data relating to availability, etc.).

The one or more actions may comprise, deploying at least one of a plurality of interventions predicted or known to increase engagement. It is advantageous to be able to predict which patients are at higher risk of non-engagement and/or drop out and therefore to differentially deploy at least one intervention with those patients, because this may therefore reduce the overall cost to the therapy provider/service of providing intervention(s), whilst at the same time achieving a reduction in non-engagement and/or drop out occurrence amongst patients (which represents a cost to the patient of non or reduced improvement or recovery). It is advantageous to be able to predict which patients are at higher risk of non-engagement and/or drop out before it occurs, rather than reacting to drop-out after it has happened, because intervention(s) deployed in advance of drop out may be more effective in increasing engagement, and therefore less likely to result in a cost to the patient. In addition, the ability to predict likelihood of non-engagement/drop-out may present a further economic benefit to the therapy provider or therapy service in pay-for-performance therapy models. Particularly, interventions predicted or known to increase engagement may be taken when the patient profile data for a patient most closely fits a reference profile describing a subtype of a psychological condition known to correspond to increased risk of drop-out or non-engagement.

The one or more actions may comprise, where the reference profile to which the patient profile data most closely fits belongs to a predetermined criterion, for example being a reference profile with a combined PHQ-9 score of 10 or less, initiating a therapy process that involves providing information to the patient via the system. In particular, the system may initiate a therapy process that does not directly (or indirectly) involve a therapist. Thus, the method may avoid unnecessary use of therapists. The avoidance of unnecessary use of therapists may be advantageous to both therapy providers/services and patients; for example therapy services may not incur unnecessary associated costs (e.g. the cost of paying therapists to provide unnecessary therapy; the further cost of reducing the availability of therapists who could otherwise be treating patients with more severe conditions), whereas patients benefit from receiving a therapy plan more appropriate to their needs, which may be beneficial in terms of e.g. convenience and/or speed of delivery.

The one or more actions taken by the method or system may include providing the output 110 as an input to a method or system performing ‘digital triage’. As explained in WO 2018/158385 A1, such a psychotherapy triage method or system may use multiple data inputs in order to take one or more actions relating to a therapy process. Thereby the reference profile to which the patient profile data most closely fits may be used as one of the multiple data inputs to the psychotherapy triage method/system.

The computer-implemented method 100 may continue being implemented to monitor the progress of the patient during or after provision of the treatment protocol 114. For example, in some instances, the prediction of the psychological condition of the patient 110 may be computed two or more times (including initially and/or during treatment) where a comparison of the prediction of psychological condition of the patient 110 at the different time points can be used as a measure of the quality of a psychological therapy.

In some instances, when a fee-for-value payment system is utilized, the quality of the psychological therapy may be used to determine the reimbursements associated with the patient's care.

### Hidden Markov Models

A Hidden Markov Model (HMM) is a statistical model in which the system being modelled is assumed to have unobserved (i.e. hidden) states. In a hidden Markov model, the state is not directly visible to the observer, but the output, dependent on the state, is visible.

For example, in the case of the assessment of depression symptoms measured using the PHQ-9 patient questionnaire, the output is the patient's answers to the PHQ-9 questionnaire which are visible to the observer, but the depression state (profile) is not. However, the probability of each hidden state (i.e. depression state) can be determined by the observed output (i.e. PHQ-9 answers).

### Network Analysis

Network theory is the study of graphs as a representation of either symmetric relations or asymmetric relations between discrete objects.

Network analysis can be used to study the relationships in complex networks, where individual elements are represented by nodes, and the connections between the elements are represented as edges (links). The relationships between any two nodes may be symmetric or asymmetric. Any two nodes may be positively correlated, negatively correlated, or not correlated with each other. The centrality of each node may be obtained: centrality indices produce rankings which seek to identify the most important nodes in a network model. The centrality of a node may be measured using a number of indices, including strength/degree (how well a node is directly connected to its neighbours), closeness (how well a node is indirectly connected to all others) and betweenness (how important a node is as a mediator in a path between two other nodes).

According to the network perspective on psychopathology, a mental disorder may be viewed as a system of interacting symptoms, with the disorder being the result of the causal interplay between symptoms. For example, excessive worry may affect concentration and lead to insomnia, which may in turn increase fatigue which also causes difficulties with concentration, a set of symptoms which may be diagnosed as an anxiety disorder, but which are also common in patients with depression. This perspective may provide therapists with specific targets of where to intervene either to prevent the development of a disorder or to treat a person who already has developed a disorder. For example, the network perspective predicts that people who have developed a symptom that is central to their depression network, are at risk of developing a full-blown episode. As such, targeting the central symptom with some kind of intervention, as soon as possible, may protect these people from progressing into clinically-significant disorder. Likewise, when treating patients who have already been diagnosed with the disorder, it may be beneficial to treatment if the strongest and weakest links in the network could be determined (i.e. which are the core symptoms, and which symptoms are of lesser importance).

Systems and corresponding computer hardware used to implement the various illustrative blocks, modules, elements, components, methods, and algorithms relative to the methods 100 described herein can include a processor configured to execute one or more sequences of instructions, programming stances, or code stored on a non-transitory, computer-readable medium. The processor can be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. In some embodiments, computer hardware can further include elements such as, for example, a memory (e.g., random access memory (RAM), flash memory, read only memory (ROM), programmable read only memory (PROM), erasable read only memory (EPROM)), registers, hard disks, removable disks, CD-ROMS, DVDs, or any other like suitable storage device or medium.

Executable sequences described herein can be implemented with one or more sequences of code (e.g., a set of instructions for implementing one or more methods 100 of the present disclosure) contained in a memory. In some embodiments, such code can be read into the memory from another machine-readable medium. Execution of the sequences of instructions contained in the memory can cause a processor to perform the process steps described herein. One or more processors in a multi-processing arrangement can also be employed to execute instruction sequences in the memory. In addition, hard-wired circuitry can be used in place of or in combination with software instructions to implement various embodiments described herein. Thus, the present embodiments are not limited to any specific combination of hardware and/or software.

As used herein, a machine-readable medium will refer to any medium that directly or indirectly provides instructions to a processor for execution.

A machine-readable medium can take on many forms including, for example, non-volatile media, volatile media, and transmission media. Non-volatile media can include, for example, optical and magnetic disks. Volatile media can include, for example, dynamic memory.

Transmission media can include, for example, coaxial cables, wire, fiber optics, and wires that form a bus. Common forms of machine-readable media can include, for example, floppy disks, flexible disks, hard disks, magnetic tapes, other like magnetic media, CD-ROMs, DVDs, other like optical media, punch cards, paper tapes and like physical media with patterned holes, RAM, ROM, PROM, EPROM and flash EPROM.

Preferably, in some instances, implementation of the methods described herein may be via a system approach where one or more of the patient profile data 102 are provided and/or updated by the patient, the service provider, or the like at a remote location (e.g., via a computer, smart phone, or other comparable device). The data may then be communicated to a central computer, which performs one or more of the analysis methods described herein. In such instances, one or more of the patient profile 102 may also be provided and/or updated at the central computer. In this example, the received data is from more than one hardware source.

Alternatively, the patient profile data 102 may be input to a central computer that performs one or more of the analysis methods described herein.

Unless otherwise indicated, all numbers expressing quantities of for example, patient variables, service variables, aggregate score, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill the art and having benefit of this disclosure.

While compositions and methods are described herein in terms of “comprising” various components or steps, the compositions and methods can also “consist essentially of” or “consist of” the various components and steps.

### EXAMPLES

### Example 1—Symptom Profiling for Depression

Data was analyzed from 4211 patients receiving IECBT for the treatment of depression, between 2012 and 2017.

PHQ-9 questionnaire responses for each patient at initial assessment and last treatment session were collected. The collated questionnaire data from all the patients at all time points available were modelled using Hidden Markov Models (HMM) implemented in R using the LMest package. Models were fitted for 1 to 16 depression states. The best fitting model was selected as the model which minimized the Bayesian Information Criteria (BIC) metric.

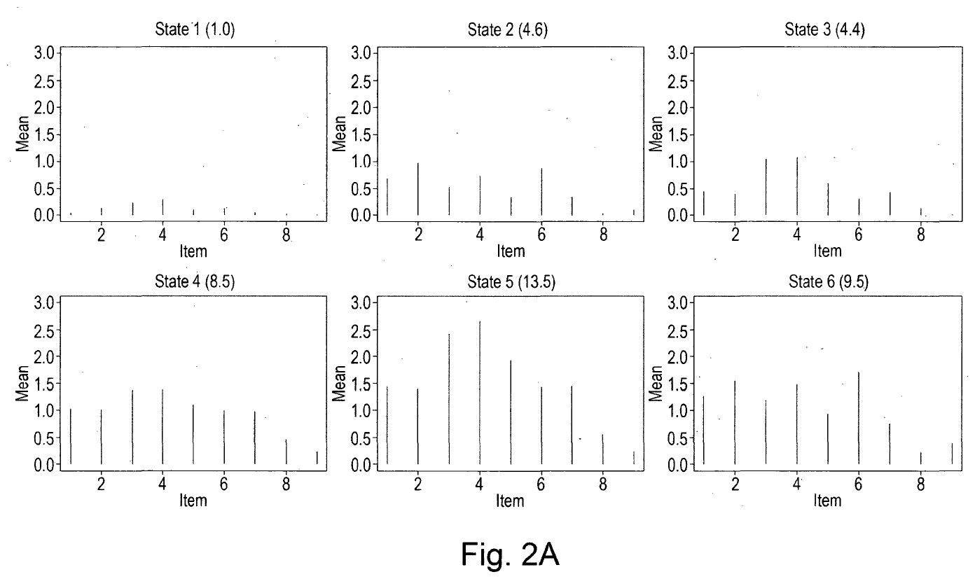

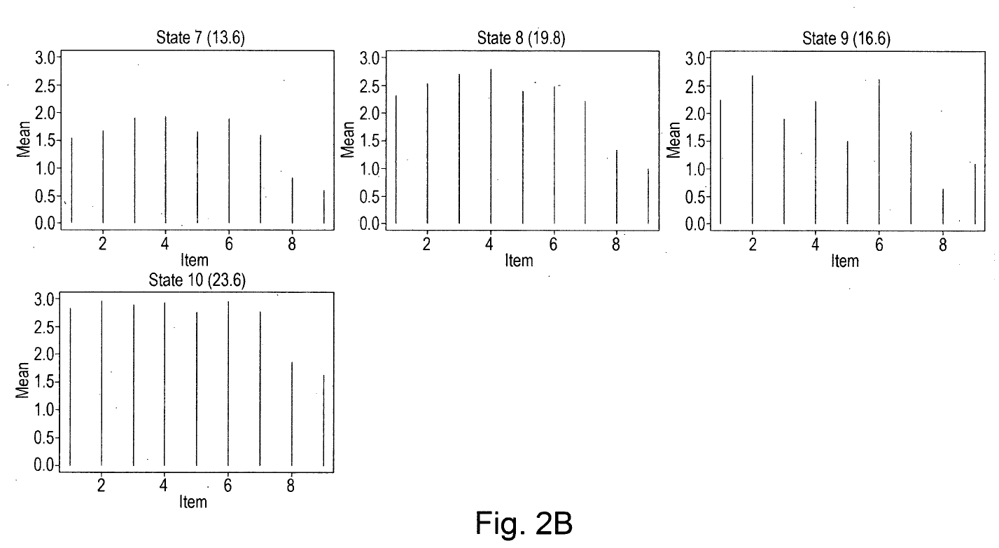

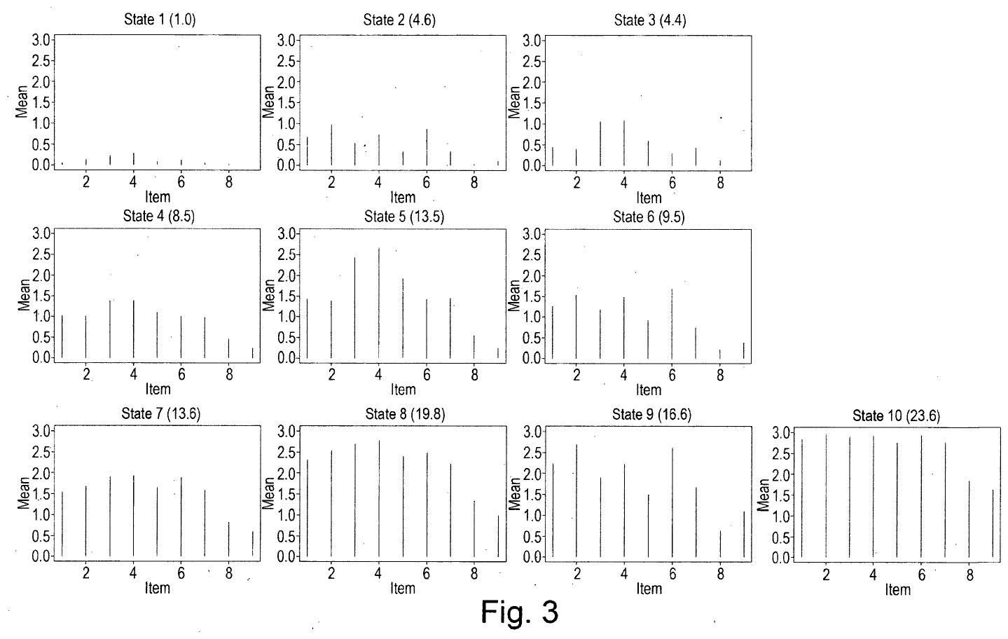

The best fitting HMM found 10 depression states (FIGS. 2 and 3) to be the optimal number to fit the data. Each state displayed a particular profile of PHQ-9 question scores, with each question (item) expressed as a mean score. Each (depression) state may be considered a reference profile. State 1 represented a fully recovered state, with all symptoms (responses to PHQ-9 questions) at floor (score <0.5). State 10 represented a maximum severity state with peak scores on all questions. States 2-9 represented intermediary severities and varying profiles. States 1 to 4 represented ‘recovered’ states, meaning that the sum of the mean scores for each of the PHQ-9 questions was less than 10—the typical threshold for determining caseness of a patient.

Some of the states appeared to display similarities of profile (i.e. the peak mean scores for those states occurred on the same questionnaire items), although with varying overall severities. For example: states 2, 6 and 9 showed the same symptom profile as each other, with peaks at questions 2, 4 and 6; states 3, 4 and 5 showed the same symptom profile as each other, with peaks at questions 3 and 4; states 7 and 8 also appeared to have the same symptom profile as each other, with peaks at questions 3, 4 and 6. These symptom profiles are identified as three distinct depression sub-types: Cognitive depression (states 2, 6 and 9), Somatic depression (States 3, 4 and 5), and Hybrid depression (States 7 and 8). The symptom profiles, relatedness of and underlying nature of the subtypes elucidated using the symptom profiler may be useful to tailor treatment to particular symptom profile(s) or subtype(s). The symptom profiler may thus be used to assist in the provision of personalized medicine.

### Example 2—State Transition Analysis of Depression States

Dataset was as per Example 1.

The patient's initial PHQ-9 questionnaire responses were fitted to the depression states as modelled in Example 1. This gave an allocated state at the start of treatment for each patient. The patient's end PHQ-9 questionnaire responses were also fitted to the depression states as modelled in Example 1, giving an allocated state at the end of treatment for each patient.

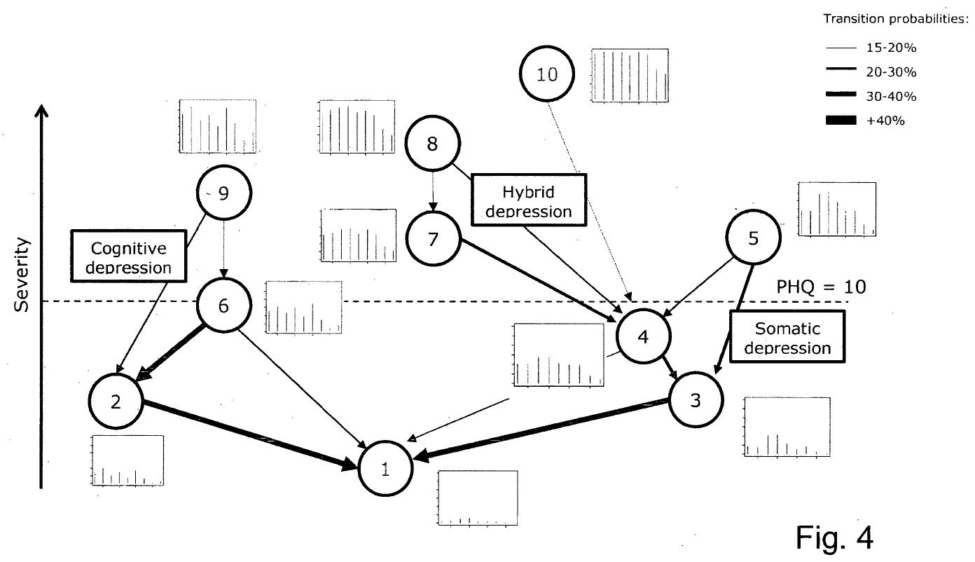

An analysis of the transitions between states was then conducted, to analyse the probability of a patient with a particular state at the start of therapy transitioning into another state at the end of treatment. This analysis was performed using Hidden Markov Models (HMM) as per Example 1, implemented in R using the LMest package. The results of this analysis are provided in FIG. 4; only transition probabilities of >15% are shown. The analysis showed that for all starting depression states, the severity of symptoms had decreased by the end of treatment. In addition, each particular depression state at the start of treatment was likely to transition into a limited number of other depression states at the end of treatment. For example, a patient allocated to state 9 based on the profile of their initial questionnaire responses had a 15-20% probability of transitioning to a state 6 profile, or a 20-30% probability of transitioning to a state 2 profile, by the end of their treatment. Alternatively, a patient allocated to state 3 based on their initial questionnaire, was more than 40% likely to be allocated to state 1 after treatment. This finding, that each starting depression state was likely to transition to only a limited number of other depression states at the end of treatment, allowed the depression states to be grouped into families of related states.

States 2, 6 and 9 were found to form one family (Group 1: Cognitive depression sub-type), states 3, 4 and 5 were found to form another family (Group 2: Somatic depression sub-type), and states 7 and 8 were found to form a third family (Group 3: Hybrid depression sub-type). States 10 and 1 were not allocated to a particular family/grouping by the network analysis.

From start to end of treatment, patients seem to transition within the same depression sub-type, and/or towards recovery, i.e. patients with cognitive depression at the start, remain in a cognitive depression state at the end of treatment, and patients with somatic depression at the start remain with somatic depression at the end of treatment, however in both cases symptom severity may be reduced.

Patients with hybrid depression appear to transition towards somatic depression, but not cognitive depression. A number of explanations for this may be possible, for example when presenting with both somatic and cognitive symptoms (hybrid depression), it may be the case that the IECBT treatment protocol used currently is more effective at dealing with cognitive symptoms, or is quicker to deal with cognitive symptoms, or perhaps the normal progression of more severe depression may be that it tends to present at earlier stages with somatic symptoms and then progress towards cognitive symptoms as the illness evolves. The symptom profiling methods may therefore be used to provide additional insights into different symptom profiles, subtypes and/or the course over time of depressive disorders. Treatment could thus be tailored to particular symptom profiles or subtype(s).

### Example 3—Core Symptom Determination

Data was analyzed from 5177 patients receiving IECBT for the treatment of depression, between 2012 and 2017. PHQ-9 and GAD-7 questionnaire responses for each patient at initial assessment were collected. Network analysis was conducted on the collated data from all patients, using a graphical lasso estimator which minimized the extended Bayesian Information Criterion, with tuning parameter gamma set to 0.5.

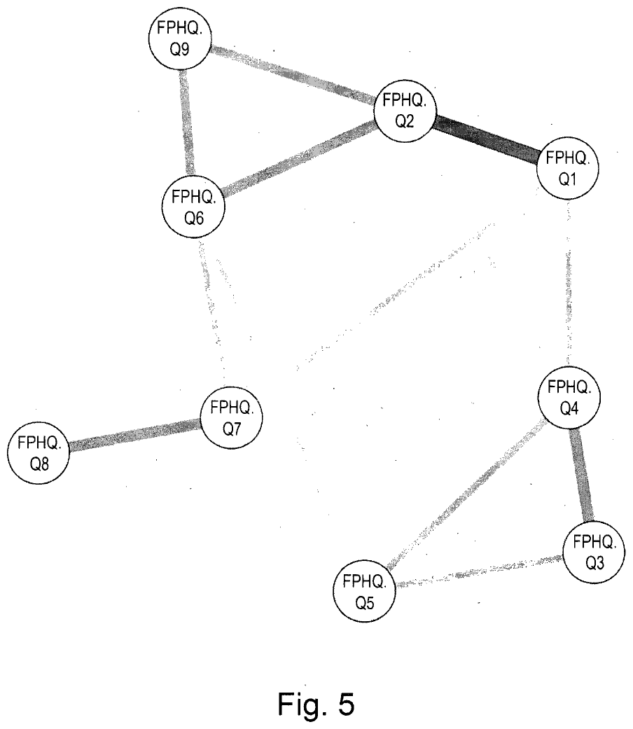

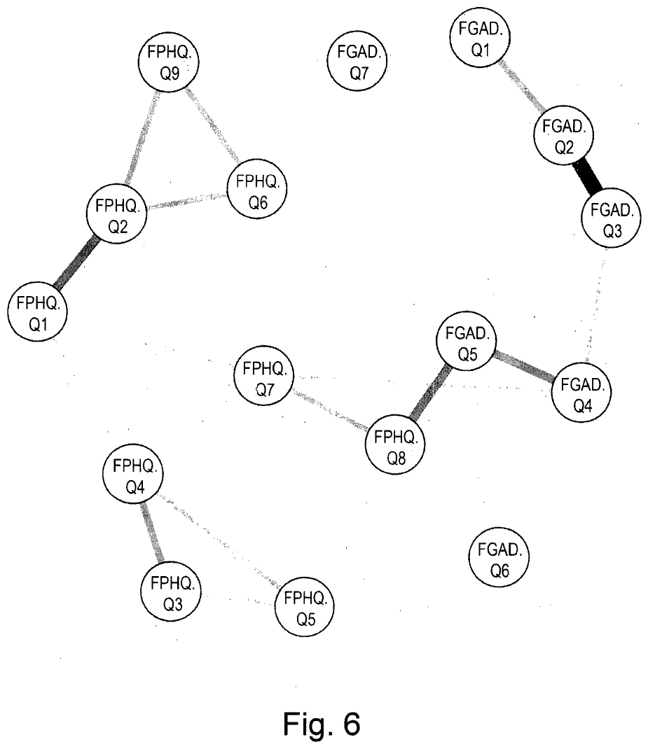

Network analysis was conducted on data relating to (i) the PHQ-9 questionnaire alone, and (ii) a combination of both the PHQ-9 and GAD-7 questionnaires. The results of these network analyses are presented in FIGS. 5 and 6. The network analyses showed that certain nodes (questionnaire items; questions; symptoms) displayed higher centrality than others. For these analyses, the best measures of centrality were strength (how well a symptom is directly connected to its neighbours) and closeness (how well a symptom is indirectly connected to all others). When analysing the PHQ-9 data alone (FIG. 5), questions 2 (FPHQ.Q2) and 4 (FPHQ.Q4) displayed the highest centrality. When analysing the PHQ-9 data in combination with the GAD-7 data (FIG. 6), PHQ-9 question 2 (FPHQ.Q2) and GAD-7 question 5 (FGAD.Q5) were the strongest, whilst the node with the highest degree of closeness was PHQ-9 question 7 (FPHQ.Q7). The data represented in FIG. 6 are also included in Table 5 below.

TABLE 5
Network analysis revealing cluster of core symptoms in depressed patients
Centrality measures per variable

| Variable | | Network | | |
|---|---|---|---|---|
| Name | Description | Betweenness | Closeness | Strength |
| FGAD.Q1 | Nervous, anxious, on | −1.054 | −1.201 | −0.874 |
| | edge | | | |
| FGAD.Q2 | Not being able to stop | −0.872 | −0.766 | 1.064 |
| | worrying | | | |
| FGAD.Q3 | Worrying about different | 0.578 | −0.205 | 1.001 |
| | things | | | |
| FGAD.Q4 | Trouble relaxing | 1.484 | 0.773 | 0.522 |
| FGAD.Q5 | Feeling restless | 1.122 | 0.970 | 1.485 |
| FGAD.Q6 | Feeling annoyed or | −1.416 | −0.789 | −1.553 |
| | irritable | | | |
| FGAD.Q7 | Something awful might | −0.510 | −0.291 | −0.750 |
| | happen | | | |
| FPHQ.Q1 | Little interest or pleasure | 0.578 | 0.978 | −0.544 |
| | in things | | | |
| FPHQ.Q2 | Down, depressed, | 1.303 | 1.149 | 1.859 |
| | hopeless | | | |
| FPHQ.Q3 | Trouble sleeping or | −1.416 | −1.597 | −0.692 |
| | sleeping too much | | | |
| FPHQ.Q4 | Tired or low energy | 0.578 | −0.507 | 0.725 |
| FPHQ.Q5 | Poor appetite or | −1.235 | −1.539 | −1.231 |
| | overeating | | | |
| FPHQ.Q6 | Feeling bad about | 0.397 | 0.764 | −0.116 |
| | yourself | | | |
| FPHQ.Q7 | Trouble concentrating | 0.940 | 1.296 | −0.355 |
| FPHQ.Q8 | Moving slowly or feeling | −0.147 | 0.983 | −0.053 |
| | fidgety | | | |
| FPHQ.Q9 | Suicidal ideation | −0.329 | −0.016 | −0.487 |

N=5,177 patients referred to leso and receiving a diagnosis of depression, from 2015 onwards; Network analysis conducted in JASP (v0.8.6, JASP team 2018).

From Table 5 and FIG. 6 it can be seen that GAD Q4 (trouble relaxing), GAD Q5 (feeling restless) and PHQ Q2 (feeling down, depressed or hopeless) show a combination of high strength, closeness and betweenness, suggesting that these are core symptoms in this group of patients.

Identifying core symptoms for each patient (or patient group; depression subtype) allows the therapist to deliver a personalized treatment plan focused on the most central symptoms in the network.

Methodology: Network model estimated using graphical lasso based on extended BIC criteria, with normalized centrality measures

Strength: measure of direct connections between a node and it's immediate neighbours, e.g. analogous to roads coming out of a town directly connecting to other towns, motorways

Closeness: measure of indirect connections between a symptom and all other symptoms in the network, e.g. analogous to roads connecting a town to all other towns in the country, even if passing through villages in between A->B->C

Betweenness: measure of how important the node is in connecting other nodes, e.g. analogous to living in a village that has a lot of through traffic, even though it's not the final destination, just people getting through to get to other destinations

The higher the centrality measures, the more important the node is in driving the network of symptoms, e.g. for depressed patients in this group, PHQ Q2 has the highest measures of centrality overall, meaning that all symptoms are causally linked to feeling down, depressed or hopeless (either caused by, or causing it).

### Example 4—Residual Sleep Symptoms

A particular treatment protocol may be designed to target particular symptoms or groups of symptoms. Targeting particular symptoms or groups of systems may be more effective in terms of treatment outcome.

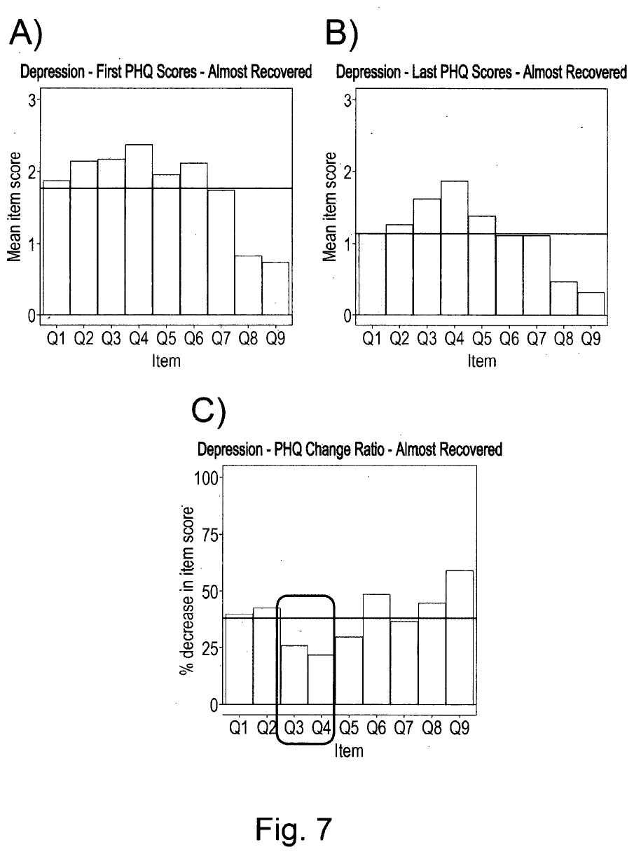

FIG. 7 shows how residual sleep symptoms may be hindering or preventing patients from achieving recovery. Symptom change over a course of treatment was measured for a particular group of patients (patients who finish a course of treatment and are ‘near misses’ at discharge; N=262 patients with depression, finishing treatment within 3 points of the threshold for recovery, in 2016 to 2017). First and last scores (obtained at the start and the end of treatment respectively) for each individual PHQ-9 question/item were recorded for each patient, and thus a PHQ (9) change ratio for each question/item was calculated (the difference between the first and last score divided by the first score, and expressed as a percentage). It is clear from FIG. 7, that symptoms related to sleep and tiredness (PHQ Q3 and PHQ Q4; set out in Table 1 above) showed the least amount of change over a course of treatment for this group of patients. Targeted interventions addressing these symptoms, before, during or after the initial course of treatment, may be beneficial for these patients, both in terms of improving these symptoms specifically, and also overall recovery.

### Example 5—Further State Transition Analysis of Depression States

Dataset was as per Example 1. However, in this example, symptom profiling for depression was conducted using data collected at multiple time-points during treatment, as opposed to just at the start and the end.

PHQ-9 questionnaire responses for each patient were obtained for all treatment sessions available (up to a maximum of 10). The collated questionnaire data from all the patients at all time points available were modelled using Hidden Markov Models (HMM) implemented in R using the LMest package. Models were fitted for 1 to 16 depression states. The best fitting model was selected as the model which minimized the Bayesian Information Criteria (BIC) metric and optimised interpretability.

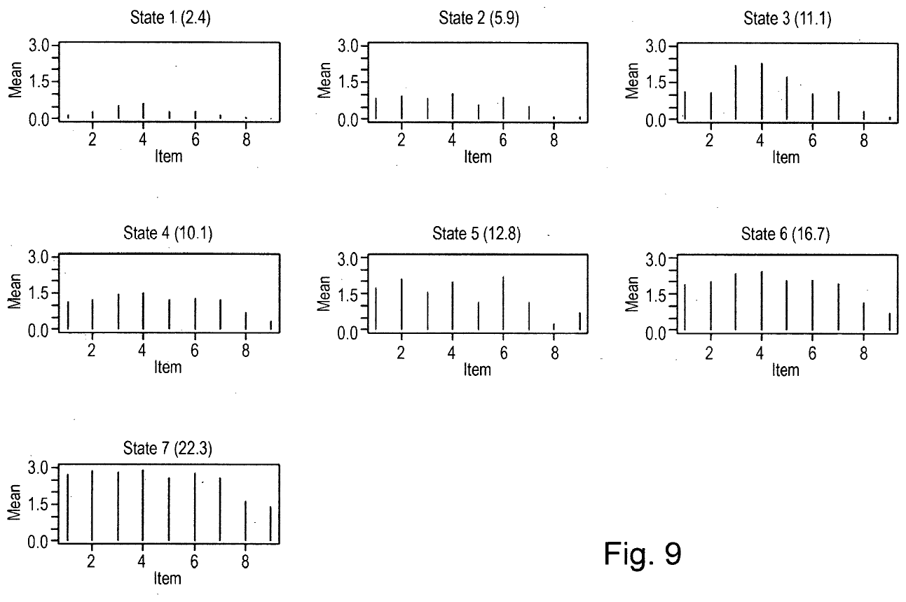

The best fitting HMM found 7 depression states (FIG. 9) to be the optimal number to fit the data whilst also optimising interpretability. Each state displayed a particular profile of PHQ-9 question scores, with each question (item) expressed as a mean score. Each (depression) state may be considered a reference profile. State 1 represented a fully recovered state, with all symptoms (responses to PHQ-9 questions) at floor (score <1). State 7 represented a maximum severity state with peak scores on all questions. States 2-6 represented intermediary severities and varying profiles. States 1 and 2 represented ‘recovered’ states, meaning that the sum of the mean scores for each of the PHQ-9 questions was less than 10—the typical threshold for determining caseness of a patient.

Some of the states appeared to display similarities of profile (i.e. the peak mean scores for those states occurred on the same questionnaire items), although with varying overall severities. For example: states 4 and 6 showed the same symptom profile as each other, with peaks at questions (items) 1 to 7; Other states however, show distinct profiles, with peak intensity for very specific items. For example, state 3 shows peak intensity for questions (items) 3 and 4, while state 5 shows peak intensity for questions (items) 2, 4 and 6. Distinct symptom profiles are identified as three distinct depression sub-types: Cognitive depression (state 5), Somatic depression (state 3), and Hybrid depression (states 4, 6 and 7).

The patient's initial PHQ-9 questionnaire responses were fitted to one of the 7 depression states as described above and in FIG. 9. This gave an allocated state at the start of treatment for each patient. The patient's PHQ-9 questionnaire responses at all time-points were also fitted to one of the 7 the depression states, giving an allocated state at each treatment session for each patient.

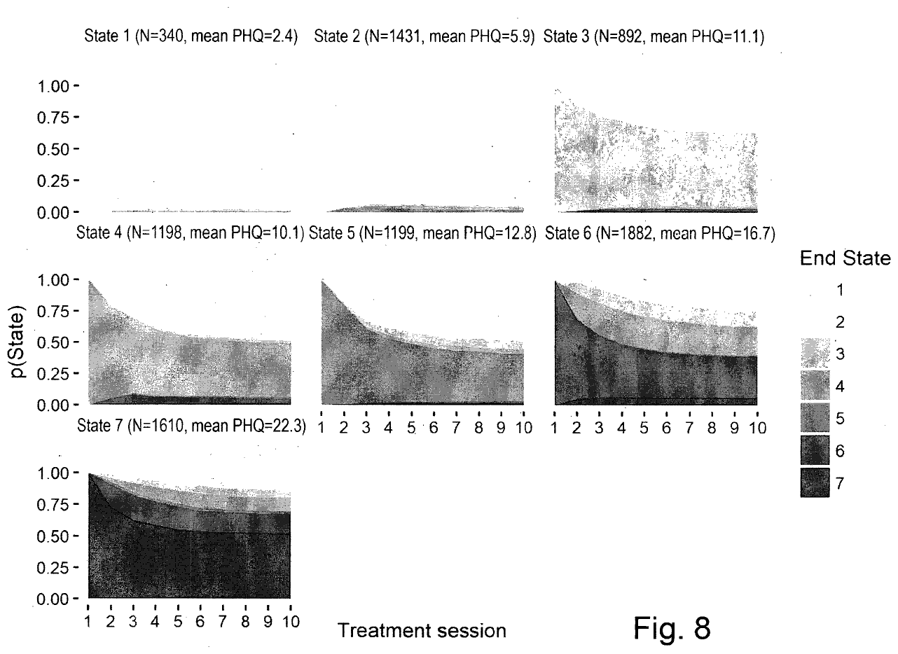

An analysis of the transitions between states was then conducted, to analyse the probability of a patient with a particular state at the start of therapy transitioning into another state during and by the end of treatment. This analysis was performed using Hidden Markov Models (HMM) as per Example 1 and above, implemented in R using the LMest package. The results of this analysis are provided in FIG. 8. The analysis showed that for all starting depression states, the severity of symptoms had decreased by the end of treatment. In addition, each particular depression state at the start of treatment was likely to transition into a limited number of other depression states at the end of treatment. For example, a patient allocated to state 5 based on the profile of their initial questionnaire responses had an approximate 25% probability of transitioning to a state 2 profile by the end of their treatment, but only circa 2% probability of transitioning to state 4 or 5% probability of transitioning to state 3. Whereas a patient allocated to state 6 based on the profile of their initial questionnaire responses had an approximate 25% probability of transitioning to a state 4 profile by the end of their treatment, but nearly negligible probability of transitioning to state 5.

Like in example 2, this finding, that each starting depression state was likely to transition to only a limited number of other depression states at the end of treatment, also allowed the depression states to be grouped into families of related states. For this model, state 5 was found to form one family (Group 1: Cognitive depression sub-type), state 3was found to form another family (Group 2: Somatic depression sub-type), and states 4, 6 and 7 were found to form a third family (Group 3: Hybrid depression sub-type). States 1 and 2 were not allocated to a particular family/grouping.

These findings accord with those presented in Examples 1 and 2, and demonstrate that the method of determining depression subtypes is robust.

From start to end of treatment, patients seem to transition within the same depression sub-type, and/or towards recovery, i.e. patients with hybrid depression at the start, remain in a hybrid depression state at the end of treatment, patients with somatic depression at the start remain with somatic depression at the end of treatment, and patients with cognitive depression at the start remain with cognitive depression at the end of treatment. However in all cases symptom severity may be reduced and the patients may transition to recovery.

The pattern was identified, where patients in a ‘somatic’ depression initial state tend to remain in a somatic depression state and not transition towards recovery with as great a likelihood as patients with similarly severe, but different, initial states. The depression subtypes identified were also correlated with other demographic factors. Patients with ‘somatic’ depression were found to be less likely to engage with treatment, more likely to suffer from long term physical comorbidities and more likely to be taking medication, than patients in other depression subtypes. Using this information, in the future patients in a somatic state of depression, for example, can be identified when entering treatment, and this information could be used to put additional measures in place, e.g. to promote patient engagement in these cases, like additional messages in between sessions, or supervisor monitoring of care.

The symptom profiling methods may therefore be used to provide additional insights into different symptom profiles, subtypes and/or the course over time of depressive disorders. Treatment could thus be tailored to particular symptom profiles or subtype(s).

### Example 6—Further Characterization of Depression Subtypes

Symptom profiling and subtyping was undertaken for 6,849 patients diagnosed with depression using traditional methods and receiving a course of internet-enabled cognitive-behavioural therapy (IECBT).

Patients completed the PHQ-9 questionnaire for depressive symptoms at presentation and prior to each therapy session. The PHQ-9 data was used as input to a hidden Markov model (HMM) to define an optimal number of depressive states. The states varied in severity and symptom profiles and patients can transition between states over a course of therapy. The states were grouped into one of 3 depression subtypes based on symptom profile—somatic, cognitive and hybrid depression. Somatic depression is characterized by high intensity of physical symptoms, including tiredness, difficulties sleeping and changes in appetite. Cognitive depression is characterized by high intensity of symptoms such as low mood, low self-esteem and high suicidal ideation. More severe hybrid depression was characterized by high intensity of both physical and psychological symptoms.

Somatic and cognitive depression subtypes were studied in depth. Classification of patients into one of these two subtypes at presentation by the HMM was blindly validated by three clinical supervisors with good inter-rater agreement (Fleiss's kappa=0.45). Demographics and response to treatment for patients presenting with either somatic or cognitive depression was then compared. Using data gathered from the therapy transcripts, the two subtypes were also compared for their response to CBT specific therapeutic features, such as agenda setting, homework setting and a variety of different change mechanisms.

Results show that despite similar severity levels at presentation, the two groups differ markedly in their response to treatment, with patients in the somatic depression subtype showing poorer engagement with treatment and poorer clinical outcomes. Patients presenting with somatic depression were also more likely to be female, take medication, and suffer from a long term physical comorbidity. Group differences in response to therapeutic features were also observed, with somatic depression patients showing a weaker response to change mechanisms. On the other hand, cognitive depression patients show a lower mean number of words per therapy session, fewer word types and lower readability.

This example represents a full characterization of depression subtypes, characterizing subtypes not only based on psychometric data, but also demographic variables and patterns of response to treatment. This data-driven, clinically validated approach represents a significant advance in characterizing depression as a heterogeneous condition. This is an important advance in the development of personalized treatment protocols for patients with depression, with the aim of improving clinical outcomes for patients with this condition and making efficiency savings for therapy services offering treatment.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described. It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A computer-implemented method of assigning a treatment protocol to a patient, comprising the steps of:
obtaining a plurality of patient profile data points relating to the patient at an initial stage of a psychotherapy process;
comparing each patient profile data point with the corresponding data point for each one of a plurality of reference profiles;
selecting from the plurality of reference profiles the reference profile to which the patient profile data most closely fits, in order to obtain a prediction of the psychological condition of the patient;
assigning a treatment protocol to the patient based on the prediction of the psychological condition of the patient;
wherein the plurality of reference profiles are determined by modelling a reference dataset comprising patient profile data relating to each of a plurality of other patients.

2. The method according to claim 1, wherein the plurality of patient profile data points comprise non-binary data.

3. The method according to claim 1, wherein the plurality of patient profile data points comprise data relating to one or more symptoms of the patient.

4. The method according to claim 1, wherein the plurality of patient profile data points comprise item scores derived from a standardised psychology questionnaire.

5. The method according to claim 4, wherein the standardised psychology questionnaire comprises the PHQ-9 and/or GAD-7 questionnaire.

6. The method according to claim 1, wherein the plurality of reference profiles comprises states determined by modelling the reference dataset using a Hidden Markov Model.

7. The method according to claim 1, wherein the patient is suffering from a mental health disorder, wherein the disorder optionally comprises a disorder selected from the group consisting of (1) depression, (2) mixed anxiety and depression, and (3) generalized anxiety disorder.

8. The method according to claim 1, wherein the prediction of the psychological condition of the patient comprises a subtype of depression, a severity of depression, or both a subtype and a severity of depression.

9. The method according to claim 1 wherein the psychotherapy process comprises internet-enabled cognitive behavioural therapy.

10. A computer-implemented method of determining subtypes of a psychological condition comprising:
obtaining patient profile data relating to each of a plurality of patients;
using a Hidden Markov Model to find a plurality of reference profiles in the patient profile data;
wherein each reference profile describes a subtype of the psychological condition.

11. The method according to claim 10 further comprising:
obtaining a plurality of patient profile data points relating to a patient at an initial stage of a psychotherapy process;
comparing each patient profile data point with the corresponding data point for each one of the plurality of reference profiles;
selecting from the plurality of reference profiles the reference profile to which the patient profile data most closely fits, in order to obtain a prediction of the psychological condition of the patient; and
assigning a treatment protocol to the patient based on the prediction of the psychological condition of the patient.

12. The method according to claim 10 further comprising:
obtaining a plurality of patient profile data points relating to a patient at an initial stage of a psychotherapy process;
comparing each patient profile data point with the corresponding data point for each one of the plurality of reference profiles;
selecting from the plurality of reference profiles the reference profile to which the patient profile data most closely fits in order to obtain an output predicting a characteristic of a condition of the patient; and
causing the system to take one or more actions relating to the psychotherapy process, wherein the one or more actions are selected based on the output.

13. The method according to claim 10 further comprising:
assigning each of the plurality of reference profiles to a family of reference profiles based on the probability of transition between each of the plurality of reference profiles;
identifying core symptoms to the family using a network analysis of individual dimensions of the patient profile data for said family; and
designing a treatment protocol to target the core symptoms for improved analysis and treatment of psychological conditions.

14. A computer-implemented method of determining families of subtypes of a psychological condition comprising:
obtaining first patient profile data relating to each of a plurality of patients at a first stage of a treatment process;
obtaining second patient profile data relating to each of the plurality of patients at a second stage of a treatment process;
obtaining combined patient profile data by combining the first patient profile data and the second patient profile data;
using a Hidden Markov Model to find a plurality of reference profiles in the combined patient profile data;
using the Hidden Markov Model to further find a probability of transition between each of the plurality of reference profiles;
assigning each of the plurality of reference profiles to a family of reference profiles based on the probability of transition between each of the plurality of reference profiles;
wherein each reference profile describes a subtype of the psychological condition.

15. The method according to claim 14, wherein the first patient profile data, the second patient profile data, or both the first and second patient profile data comprise data relating to one or more symptoms of each of the plurality of patients.

16. The method according to claim 10, the first patient profile data, the second patient profile data, or both the first and second patient profile data comprise data derived from a standardised psychology questionnaire, optionally wherein the standardised psychology questionnaire is selected from the PHQ-9 questionnaire or the GAD-7 questionnaire.

17. The method according to claim 10, wherein the psychological condition comprises depression.

18. (canceled)

19. (canceled)

20. (canceled)

21. (canceled)
